(19) United States
(12) Patent Application Publication
(10) Pub. No.: US 2025/0331858 A1
(43) Pub. Date: Oct. 30, 2025

(54) DEVICES AND METHODS FOR PROVIDING PASSAGE BETWEEN HEART CHAMBERS

(71) Applicant: V-Wave Ltd., Caesarea (IL)

(72) Inventors: Tamir BEN-DAVID, Tel Aviv (IL); Neal EIGLER, Agoura Hills, CA (US); Nir NAE, Binyamina (IL); Lior ROSEN, Zikhron Ya'akov (IL); Erez ROZENFELD, Shoham (IL); James S. WHITING, Los Angeles, CA (US)

(73) Assignee: V-Wave Ltd., Caesarea (IL)

(21) Appl. No.: 19/257,031

(22) Filed: Jul. 1, 2025

Related U.S. Application Data
(63) Continuation of application No. 18/458,642, filed on Aug. 30, 2023, now Pat. No. 12,349,912.
Application No. 18/458,642 is a continuation of application No. 16/963,139, filed on Jul. 17, 2020, now Pat. No. 11,744,589.
(60) Provisional application No. 62/619,748, filed on Jan. 20, 2018.

(51) Int. Cl. A61B 17/11; A61B 17/00

(52) U.S. Cl. CPC A61B 17/11; A61B 17/0057; A61B 2017/00022; A61B 2017/00084; A61B 2017/00106; A61B 2017/00243; A61B 2017/00358; A61B 2017/00592; A61B 2017/00606; A61B 2017/00623; A61B 2017/1107; A61B 2017/1139; A61B 2562/0247

(57) ABSTRACT

A device for providing a passage between a first and second heart chamber is provided. The device includes a middle region having first and second ends, a lumen extending therethrough having a longitudinal axis, a first end region coupled to the first end, and a second end region coupled to the second end. The first end region may be delivered in the first heart chamber in a compressed state and transitioned to a deployed state, the first end region being deformable such that portions of the first end region are expandable to different angles relative to the longitudinal axis. The second end region may be delivered in the second heart chamber in a compressed state and transitioned to a deployed state therein, the second end region being deformable such that portions of the second end region are expandable to different angles relative to the longitudinal axis.

### CROSS-REFERENCE TO RELATED APPLICATIONS

This application is a continuation application of U.S. patent application Ser. No. 18/458,642, filed Aug. 30, 2023, now U.S. Pat. No. 12,349,912, which is a continuation application of U.S. patent application Ser. No. 16/963,139, filed Jul. 17, 2020, now U.S. Pat. No. 11,744,589, which is a national phase application under 35 U.S.C. § 371 of PCT/IB2019/050452, filed Jan. 19, 2019, which claims priority to U.S. Patent Provisional Application Ser. No. 62/619,748, filed Jan. 20, 2018, the entire contents of each of which are incorporated by reference herein.

### FIELD OF THE INVENTION

This application generally relates to percutaneously placed implants and methods for providing a passage between body cavities, e.g., heart chambers, to address pathologies such as heart failure (“HF”), myocardial infarction (“MI”) and pulmonary arterial hypertension (“PAH”), and to provide access to a surgeon's tool between the heart chambers.

### BACKGROUND OF THE INVENTION

For a number of medical conditions, there is benefit in creating and/or maintaining a passage between two body cavities. Such a passage is typically used in catheterization procedures where the catheter is delivered through a patient's vasculature. In some catheterization procedures, there is a benefit in moving from one cavity to another cavity by creating a passage. For example, such a passage may be formed between the right side of the heart and the left side of the heart, e.g., between the right atrium toward the left atrium, where clinical procedures are done on the left side of the heart using an entry from the right side of the heart. Such clinical procedures include, e.g., AV nodal ablation in the left atrium or left ventricle and mitral valve repair activities.

In addition, a passage may be created and maintained in a heart wall between two heart chambers for housing a shunt for redistributing blood from one heart chamber to another to address pathologies such as HF, MI, and PAH. Heart failure is the physiological state in which cardiac output is insufficient to meet the needs of the body or to do so only at a higher filling pressure. There are many underlying causes of HF, including myocardial infarction, coronary artery disease, valvular disease, hypertension, and myocarditis. Chronic heart failure is associated with neurohormonal activation and alterations in autonomic control. Although these compensatory neurohormonal mechanisms provide valuable support for the heart under normal physiological circumstances, they also play a fundamental role in the development and subsequent progression of HF.

HF is generally classified as either systolic heart failure (“SHF”) or diastolic heart failure (“DHF”). In SHF, the pumping action of the heart is reduced or weakened. A common clinical measurement is the ejection fraction, which is a function of the blood ejected out of the left ventricle (stroke volume) divided by the maximum volume in the left ventricle at the end of diastole or relaxation phase. A normal ejection fraction is greater than 50%. Systolic heart failure generally causes a decreased ejection fraction of less than 40%. Such patients have heart failure with reduced ejection fraction (“HFrEF”). A patient with HFrEF may usually have a larger left ventricle because of a phenomenon called “cardiac remodeling” that occurs secondarily to the higher ventricular pressures.

In DHF, the heart generally contracts normally, with a normal ejection fraction, but is stiffer, or less compliant, than a healthy heart would be when relaxing and filling with blood. Such patients are said to have heart failure with preserved ejection fraction (“HFpEF”). This stiffness may impede blood from filling the heart and produce backup into the lungs, which may result in pulmonary venous hypertension and lung edema. HFpEF is more common in patients older than 75 years, especially in women with high blood pressure.

Both variants of HF have been treated using pharmacological approaches, which typically involve the use of vasodilators for reducing the workload of the heart by reducing systemic vascular resistance, as well as diuretics, which inhibit fluid accumulation and edema formation, and reduce cardiac filling pressure. No pharmacological therapies have been shown to improve morbidity or mortality in HFpEF whereas several classes of drugs have made an important impact on the management of patients with HFrEF, including renin-angiotensin antagonists, beta blockers, and mineralocorticoid antagonists. Nonetheless, in general, HF remains a progressive disease and most patients have deteriorating cardiac function and symptoms over time. In the U.S., there are over 1 million hospitalizations annually for acutely worsening HF and mortality is higher than for most forms of cancer.

In more severe cases of HFrEF, assist devices such as mechanical pumps are used to reduce the load on the heart by performing all or part of the pumping function normally done by the heart. Chronic left ventricular assist devices (“LVAD”), and cardiac transplantation, often are used as measures of last resort. However, such assist devices typically are intended to improve the pumping capacity of the heart, to increase cardiac output to levels compatible with normal life, and to sustain the patient until a donor heart for transplantation becomes available. Such mechanical devices enable propulsion of significant volumes of blood (liters/min), but are limited by a need for a power supply, relatively large pumps, and pose a risk of hemolysis, thrombus formation, and infection. Temporary assist devices, intra-aortic balloons, and pacing devices have also been used.

Various devices have been developed using stents to modify blood pressure and flow within a given vessel, or between chambers of the heart. For example, U.S. Pat. No. 6,120,534 to Ruiz is directed to an endoluminal stent for regulating the flow of fluids through a body vessel or organ, for example, for regulating blood flow through the pulmonary artery to treat congenital heart defects. The stent may include an expandable mesh having lobed or conical portions joined by a constricted region, which limits flow through the stent. The mesh may comprise longitudinal struts connected by transverse sinusoidal or serpentine connecting members. Ruiz is silent on the treatment of HF or the reduction of left atrial pressure.

U.S. Pat. No. 6,468,303 to Amplatz et al. describes a collapsible medical device and associated method for shunting selected organs and vessels. Amplatz describes that the device may be suitable to shunt a septal defect of a patient's heart, for example, by creating a shunt in the atrial septum of a neonate with hypoplastic left heart syndrome (“HLHS”). That patent also describes that increasing mixing of pulmonary and systemic venous blood improves oxygen saturation, and that the shunt may later be closed with an occluding device. Amplatz is silent on the treatment of HF or the reduction of left atrial pressure, as well as on means for regulating the rate of blood flow through the device.

Implantable interatrial shunt devices have been successfully used in patients with severe symptomatic heart failure. By diverting or shunting blood from the left atrium (“LA”) to the right atrium (“RA”), the pressure in the left atrium is lowered or prevented from elevating as high as it would otherwise (left atrial decompression). Such an accomplishment would be expected to prevent, relieve, or limit the symptoms, signs, and syndromes associated of pulmonary congestion. These include severe shortness of breath, pulmonary edema, hypoxia, the need for acute hospitalization, mechanical ventilation, and death.

Shunt flow is generally governed by the pressure gradient between the atria and the fluid mechanical properties of the shunt device. The latter are typically affected by the shunt's geometry and material composition. For example, the general flow properties of similar shunt designs have been shown to be related to the mean interatrial pressure gradient and the effective orifice diameter.

Percutaneous implantation of interatrial shunts generally requires transseptal catheterization immediately preceding shunt device insertion. The transseptal catheterization system is placed from an entrance site in the femoral vein, across the interatrial septum in the region of fossa ovalis (“FO”), which is the central and thinnest region of the interatrial septum. The FO in adults is typically 15-20 mm in its major axis dimension and ≤3 mm in thickness, but in certain circumstances may be up to 10 mm thick. LA chamber access may be achieved using a host of different techniques familiar to those skilled in the art, including but not limited to: needle puncture, stylet puncture, screw needle puncture, and radiofrequency ablation. The passageway between the two atria is dilated to facilitate passage of a shunt device having a desired orifice size. Dilation generally is accomplished by advancing a tapered sheath/dilator catheter system or inflation of an angioplasty type balloon across the FO. This is the same general location where a congenital secundum atrial septal defect (“ASD”) would be located.

U.S. Patent Publication No. 2005/0165344 to Dobak, III describes apparatus for treating heart failure that includes a tubular conduit having an emboli filter or valve, the device configured to be positioned in an opening in the atrial septum of the heart to allow flow from the left atrium into the right atrium. Dobak discloses that shunting of blood may reduce left atrial pressures, thereby preventing pulmonary edema and progressive left ventricular dysfunction, and reducing LVEDP. Dobak describes that the device may include deployable retention struts, such as metallic arms that exert a slight force on the atrial septum on both sides and pinch or clamp the device to the septum.

In addition, following implantation of a shunt device within a heart wall, tissue ingrowth including an endothelial layer or neointima layer typically forms on the device, thereby inhibiting thrombogenicity of the shunt device, and narrowing the size of the passage through the device. U.S. Patent Publication No. 2013/0178784 to McNamara describes an adjustable pressure relief shunt that may be expanded, e.g., via an inflation balloon. McNamara describes that the tubular body of the shunt may be plastically deformable and that the size of the shunt may be repeatedly adjusted responsive to measurements of the patient's physiological parameters. McNamara does not describe adjusting the size of the shunt to accommodate specifically sized clinical procedure tools used by the surgeon.

It would therefore be desirable to provide device and methods for adjusting the size of a passage through a device in situ to responsive to the clinical procedures performed by the surgeon.

In addition, it would further be desirable to provide device and methods for adjusting the cross-sectional area at the inlet and outlet ends of the device in situ.

It further may be beneficial to create passages between the venous blood vessels and the arterial blood vessels, between the venous blood vessels and the heart chambers, or between arterial blood vessels and the heart chambers. Following the catheterization procedure such passages are normally left open or sealed by special sealing devices such as an atrial septal occluder.

### SUMMARY OF THE INVENTION

In view of the foregoing drawbacks of previously-known shunt devices, an adjustable passage device constructed in accordance with the principles of the present invention provides a more durable configuration that maintains luminal patency for extended periods of time. The inventive adjustable passage devices further enable particular selection of desired passage diameters for permitting various sized catheterization tools therethrough, as well as inlet and outlet diameters and angles so as to conform to a variety of tissue geometries between adjacent body cavities, thereby securely anchoring the passage device within the tissue.

In accordance with one aspect of the present invention, a device for providing a passage between a first heart chamber and a second heart chamber is provided. The device includes a middle region having first and second ends, a lumen extending therethrough, and a longitudinal axis aligned with the lumen, a first end region coupled to the first end, and a second end region coupled to the second end. The first end region may be delivered in the first heart chamber in a compressed delivery state and transitioned to a deployed state therein, the first end region being selectively deformable such that selected portions of the first end region are expandable to different angles relative to the longitudinal axis. In addition, the second end region may be delivered in the second heart chamber in a compressed delivery state and transitioned to a deployed state therein, the second end region being selectively deformable such that selected portions of the second end region are expandable to different angles relative to the longitudinal axis. At least one of the selected portions of the first or second end regions are expandable to an angle between zero and 90 degrees relative to the longitudinal axis of the device. The first and second end regions are constructed to anchor the middle region within a heart wall between the first heart chamber and the second heart chamber when in the expanded deployed state.

The first and second end region may be formed of a plastically deformable material. In addition, the first and second end regions may be transitionable from the compressed delivery state to the expanded deployed state via different sized non-compliant balloons. In accordance with one aspect of the present invention, the first and second end regions include a plurality of support arms extending from the middle region, the plurality of support arms coupled circumferentially along outer edges of the middle region of the device. In accordance with another aspect of the present invention, the first and second end regions are integrally formed with the middle region, such that the first and second end regions and the middle region are formed of a plurality of longitudinal struts interconnected by a plurality of circumferential sinusoidal struts. Accordingly, at least one of the first or second end regions has at least one of a conical or bell shape.

Moreover, the middle region is adjustable from a first state having a first diameter to a second state having a second diameter different from the first diameter. For example, the middle region may be formed of a plastically deformable material and/or an expandable mesh tube. The second diameter may be larger than the first diameter, or it may be smaller than the first diameter. The middle region may be adjusted from the first state to the larger second state via an inflatable balloon catheter. For example, the balloon catheter may be a dog bone shape or a quadrilateral dog bone shape. In addition, the passage device may include one or more sensors for measuring blood flow through the passage between the first heart chamber and the second heart chamber, such that the middle region may be adjusted from the first state to the second state responsive to the measured blood flow.

The middle region of the device further may be coupled to a medical device to thereby anchor the medical device within the heart wall between the first heart chamber and the second heart chamber. For example, the medical device may be at least one of a septal occluder, an open atrial septal shunt, a valved atrial septal shunt, a left atrial blood pressure sensor, or a blood pump. In accordance with one aspect of the present invention, the first heart chamber is a left atrium and the second heart chamber is a right atrium, such that the device permits blood flow through the passage between the left atrium and the right atrium.

In accordance with another aspect of the present invention, a method for providing a passage between a first heart chamber and a second heart chamber is provided. The method includes selecting a device having a first end region, a second end region, and a middle region extending between the first and second end regions, the middle region having a lumen for providing the passage between the first heart chamber and the second heart chamber. The method further includes delivering the device in a compressed delivery state within a heart wall of a patient such that the first end region is disposed within the first heart chamber, the second end region is disposed within the second heart chamber, and the middle region is positioned within the heart wall. In addition, the method includes expanding the first end region from the compressed delivery state to an expanded deployed state such that selected portions of the first end region have different angles relative to a longitudinal axis of the device, and expanding the second end region from the compressed delivery state to an expanded deployed state such that selected portions of the second end region have different angles relative to a longitudinal axis of the device, thereby providing the passage through the lumen of the middle region between the first heart chamber and the second heart chamber. Further, the method may include adjusting an angle of the first end region relative to the longitudinal axis of the device, and adjusting an angle of the second end region relative to the longitudinal axis of the device to achieve a predetermined flowrate across the passage between the first heart chamber and the second heart chamber.

### BRIEF DESCRIPTION OF DRAWINGS

FIGS. 1A and 1B are cross-sectional views of an exemplary passage device in an expanded deployed state and a compressed delivery state, respectively, constructed in accordance with the principles of the present invention.

FIGS. 2A to 2D illustrate various expanded deployed configurations of the device of FIG. 1A disposed within a tissue between two heart chambers in accordance with the principles of the present invention.

FIG. 3 is a front view of the device of FIG. 1A disposed within a heart wall between two heart chambers.

FIG. 4A illustrates an alternative exemplary passage device constructed in accordance with the principles of the present invention.

FIG. 4B illustrates the device of FIG. 4A having a sensor device disposed therein.

FIG. 5 illustrates another alternative exemplary passage device constructed in accordance with the principles of the present invention.

FIGS. 6 and 7 depict the device of FIG. 5 having various alternative embodiments of a shunt disposed therein in accordance with the principles of the present invention.

FIGS. 8A and 8B depict the device of FIG. 5 having a stent subsequently disposed therein in accordance with the principles of the present invention.

FIGS. 9A to 9C are cross-sectional views of an embodiment of a passage device wherein the passage through the device is adjusted in situ in accordance with the principles of the present invention.

FIGS. 10A to 10C are cross-sectional views of an alternative embodiment of a passage device wherein the passage through the device is adjusted in situ in accordance with the principles of the present invention.

FIGS. 11A to 11C are cross-sectional views of another alternative embodiment of a passage device wherein the passage through the device is adjusted in situ in accordance with the principles of the present invention.

FIG. 12A illustrates an exemplary dog bone shaped balloon catheter, in a deflated state, for transitioning the end regions of a passage device from a compressed delivery state to an expanded deployed state, constructed in accordance with the principles of the present invention.

FIG. 12B illustrates the exemplary dog bone shaped balloon catheter of FIG. 12A in an inflated state.

FIG. 12C illustrates another exemplary dog-bone shaped catheter constructed in accordance with the principles of the present invention.

FIGS. 13A to 13C illustrate the steps of using the exemplary dog bone shaped balloon catheter of FIG. 12A to transition the end regions of a passage device from a compressed delivery state to an expanded deployed state.

FIGS. 14A and 14B illustrate the steps of using the exemplary dog bone shaped balloon catheter of FIG. 12C to transition the end regions of a passage device from a compressed delivery state to an expanded deployed state.

FIG. 15A illustrates an exemplary quadrilateral dog bone shaped balloon catheter for transitioning the end regions of a passage device from a compressed delivery state to an expanded deployed state, constructed in accordance with the principles of the present invention.

FIG. 15B illustrates the exemplary quadrilateral dog bone shaped balloon catheter of FIG. 15A being used to transition the end regions of a passage device from a compressed delivery state to an expanded deployed state.

FIGS. 16A to 16C illustrate the steps of using an exemplary balloon catheter to adjust the size of the passage of a passage device in accordance with the principles of the present invention.

FIGS. 17A and 17B illustrate the steps of using a snare to adjust the size of the passage of a passage device in accordance with the principles of the present invention.

FIGS. 18A to 18C illustrate the steps of subsequently placing a medical device within the device of FIG. 1A in accordance with the principles of the present invention.

FIGS. 19A to 19D illustrate the steps of subsequently placing a medical device having expandable ends within the device of FIG. 1A in accordance with the principles of the present invention.

FIG. 20 is a flow chart of an exemplary method for transitioning the end regions of a passage device from a compressed delivery state to an expanded deployed state in accordance with the principles of the present invention.

### DETAILED DESCRIPTION OF THE PREFERRED EMBODIMENTS

Devices are provided for providing a passage between adjacent body cavities, e.g., hearth chambers, within a patient. The diameter of the passage through the device may be adjusted accordingly responsive to the needs of the clinical procedure through the passage. In addition, the angles and cross-sectional areas of the proximal and distal end regions of the device may be independently selected to secure the device within the tissue, e.g., heart wall, and to selectively control the flowrate through the device responsive to the pressure gradient across the device. Further, the device may be designed to anchor an additional medical device within the heart wall, such as a septal occluder, an open atrial septal shunt, a valved atrial septal shunt, a left atrial blood pressure sensor, or a blood pump.

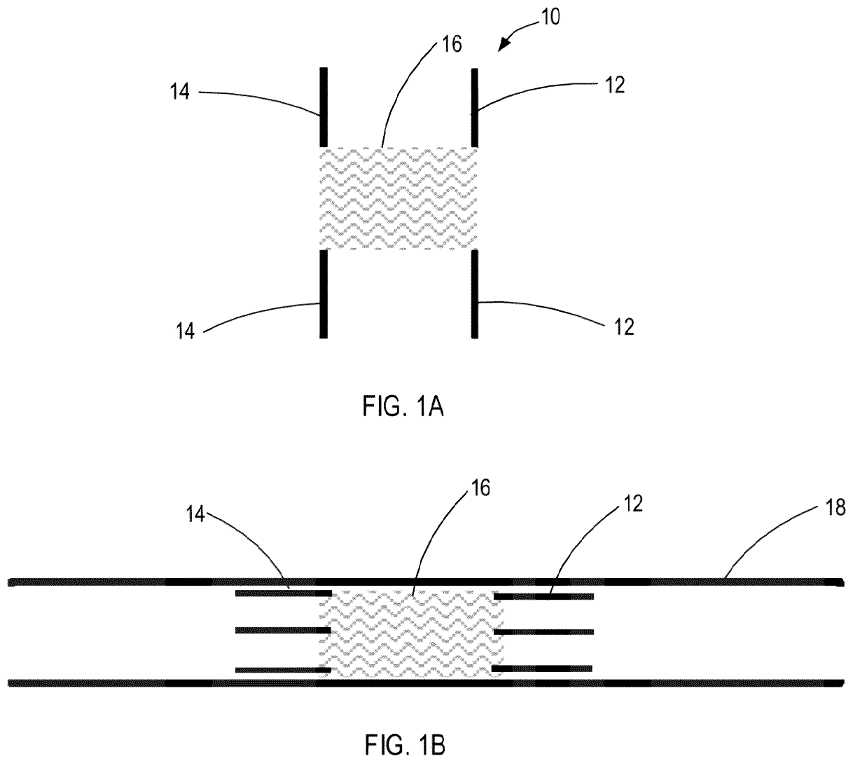

Referring now to FIGS. 1A and 1B, an exemplary passage device is provided. Passage device 10 includes first end region 12, second end region 14, and middle region 16 extending between first end region 12 and second end region 14. First end region 12 and second end region 14 may be formed of an expandable material such that first end region 12 and second end region 14 are transitionable between an expanded deployed state as shown in FIG. 1A, and a compressed delivery state as shown in FIG. 1B. As illustrated in FIG. 1B, passage device 10 may be disposed within sheath 18 in the compressed delivery state for percutaneously delivery to the target site.

Middle region 16 of passage device 10 may be formed from a mesh tube of a material having plastic properties, e.g., Cobalt Chromium. Accordingly, middle region 10 also may be transitionable between a compressed delivery state and an expanded deployed state. For example, Cobalt Chromium mesh tube may first undergo elastic deformation subject to stress that is lower than its yield strength prior to plastic deformation. Alternatively, the Cobalt Chromium mesh tube may receive a designated heat treatment to optimize performance (with respect to plasticity) for its specific application and/or desired geometry. Upon delivery and deployment of passage device 10 at the target tissue, e.g., heart wall, the diameter of the lumen of middle region 16 may be further adjusted to a desired size. Specifically, the plastically deformable material of middle region 16 may be expanded to a desired size such that the plastically deformable material maintains the desired size upon removal of the expansion force applied to middle region 16. For example, different catheterization procedures may require tools of various sizes, and thus, middle region 16 of passage device 10 may be adjusted to have a diameter sufficient to permit a desired tool to pass therethrough. In addition, middle region 16 may be compressed to a smaller desired size such that the plastically deformable material maintains the smaller desired size upon removal of the compression force applied to middle region 16, e.g., via a snare.

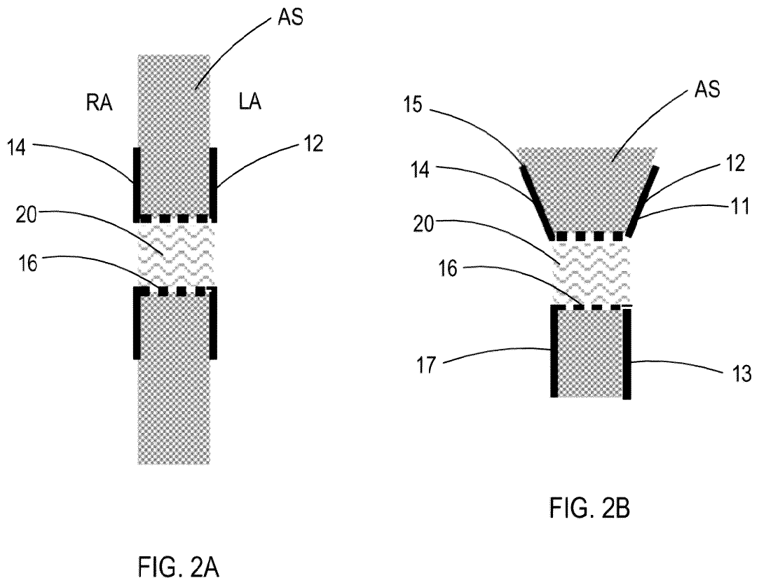

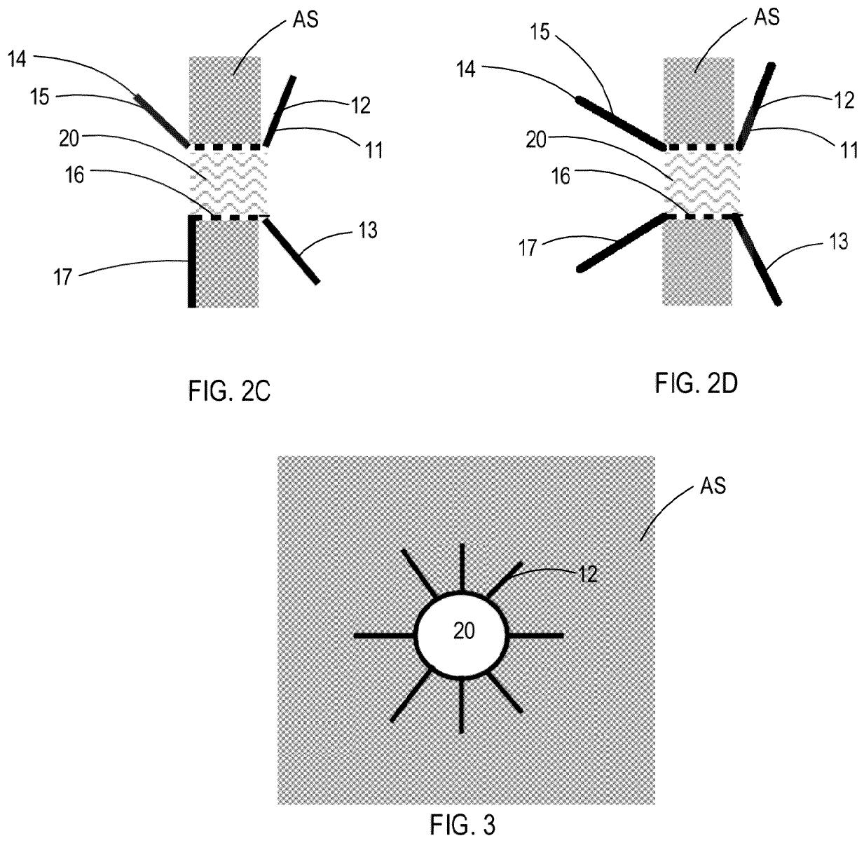

Referring now to FIGS. 2A to 2D, passage device 10 may be deployed within a heart wall of the patient, e.g., atrial septum AS. Accordingly, as shown in FIG. 2A, when middle region 16 is positioned within an opening in atrial septum AS, first end portion 12 of passage device 10 may be disposed within left atrium LA and second end portion 14 of passage device 10 may be disposed within right atrium RA, thereby providing passage 20 through middle region 16 in fluid communication with left atrium LA and right atrium RA. Moreover, as shown in FIG. 2A, first end region 12 and second end region 14 may be expanded such that they each extend into their respective atria at an angle of 90 degrees relative to the longitudinal axis of passage device 10. Accordingly, first end region 12 and second end region 14 may extend parallel along the wall of the atrial septum AS. This permits first end region 12 and second end region 14 to anchor middle region 16 within atrial septum AS.

As shown in FIG. 2B, first portion 11 of first end region 12 may be expanded such that it extends into the left atrium at an angle between 45 degrees and 90 degrees relative to the longitudinal axis of passage device 10, whereas second portion 13 of first end region 12 is expanded such that it extends into the left atrium at an angle of 90 degrees relative to the longitudinal axis of passage device 10, to thereby conform to the shape of atrial septum AS from within the left atrium as depicted in FIG. 2B. Similarly, first portion 15 of second end region 14 may be expanded such that it extends into the right atrium at an angle between 45 degrees and 90 degrees relative to the longitudinal axis of passage device 10, whereas second portion 17 of second end region 14 is expanded such that it extends into the right atrium at an angle of 90 degrees relative to the longitudinal axis of passage device 10, to thereby conform to the shape of atrial septum AS from within the right atrium as depicted in FIG. 2B. These expansions may be carried out via various sized balloon catheters, and/or a balloon catheter having various expandable portions, each independently inflatable to a desired inflation size. As will be understood by a person having ordinary skill in the art, first portion 11 and second portion 13 may be expanded such that they extend into the left atrium at any angle relative to the longitudinal axis of passage device 10 as may be required to anchor passage device 10 within atrial septum AS. For example, first portion 11 and second portion 13 may be expanded such that they extend into the left atrium at an angle between zero and 90 degrees, or even greater than 90 degrees.

Referring now to FIG. 2C, first end region 12 is selectively deformable such that selected portions of first end region 12 are expandable to different angles relative to the longitudinal axis of passage device 10, and first end region 12 is selectively deformable such that selected portions of first end region 12 are expandable to different angles relative to the longitudinal axis of passage device 10. For example, first portion 11 of first end region 12 may be expanded such that it extends into the left atrium at an angle between 45 degrees and 90 degrees relative to the longitudinal axis of passage device 10, whereas second portion 13 of first end region 12 is expanded such that it extends into the left atrium at an angle of 45 degrees relative to the longitudinal axis of passage device 10. Further, first portion 15 of second end region 14 may be expanded such that it extends into the right atrium at an angle of 45 degrees relative to the longitudinal axis of passage device 10, whereas second portion 17 of second end region 14 is expanded such that it extends into the right atrium at an angle of 90 degrees relative to the longitudinal axis of passage device 10. The selective expansion of selected portions of first end region 12 and second end region 14 may be carried out via different sized non-compliant balloons, and/or a balloon catheter having various expandable portions, each independently inflatable to a desired inflation size.

Referring now to FIG. 2D, first end region 12 may be transitioned from a contracted delivery state to an expanded deployed state in which first end region 12 extends into the left atrium at a first angle relative to a longitudinal axis of passage device 10, whereas second end region 14 is transitioned from a contracted delivery state to an expanded deployed state in which second end region 14 extends into the right atrium at a second angle relative to the longitudinal axis of passage device 10 that is different from the first angle of first end region 12. Therefore, the angles of inlet, e.g., first end region 12, and the outlet, e.g., second end region 14, of passage device 10, may be selected to precisely control and optimize the flowrate of blood through passage 20 of passage device 10 when passage device 10 is utilized as a shunt between the left and right atria, thereby changing the coefficient of discharge (“CD”) of passage device 10, i.e., the ratio between the effective orifice area to the true orifice area. For example, if first end region 12 is expanded to 90 degrees relative to the longitudinal axis of passage device 10 to match the plane of the atrial septum, the CD is about 0.65, while if first end region 12 is expanded to 45 degrees relative to the longitudinal axis of passage device 10, the CD may be around 0.9. In addition, the inlet and outlet angles may be selected to control and optimize flow dynamics with respect to minimization of turbulence and flow stagnation across passage device 10.

As shown in FIG. 2D, first portion 11 and second portion 13 of first end region 12 may be expanded such that they extend into the left atrium at an angle between 45 degrees and 90 degrees relative to the longitudinal axis of passage device 10, whereas first portion 15 and second portion 17 of second end region 14 are expanded such that they extend into the right atrium at an angle between zero and 45 degrees relative to the longitudinal axis of passage device 10. The expansion of first end region 12 and second end region 14 may be carried out via different sized non-compliant balloons, and/or a balloon catheter having various expandable portions, each independently inflatable to a desired inflation size.

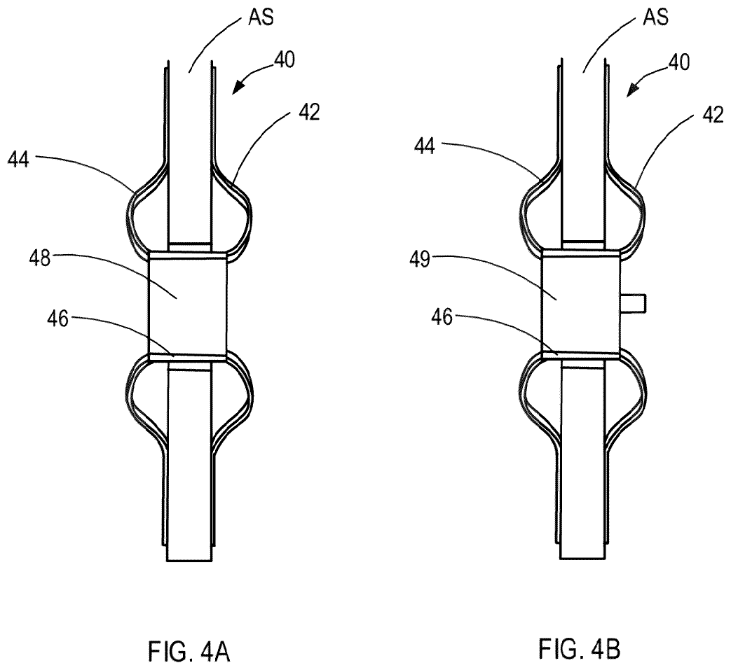

As illustrated in FIG. 3, which depicts passage device 10 positioned within atrial septum AS from within the left atrium, first end region 12 may include a plurality of support arms coupled circumferentially along outer edges of the middle region of passage device 10. For example, as shown in FIG. 4A, the passage device may be constructed similar to the differential pressure regulating device disclosed in U.S. Pat. No. 8,070,708 to Rottenberg, assigned to the assignee of the instant application, the entire contents of which is incorporated herein by reference. Specifically, passage device 40 of FIG. 4A includes first end portion 42 extending from a proximal edge of middle region 46 into the left atrium, and second end portion 44 extending from a distal edge of middle region 46 into the right atrium. As shown in FIG. 4A, first end region 42 includes a plurality of support arms that extend radially outward from the proximal end of middle region 46, curving away from middle region 46 into the left atrium, then curving back toward atrial septum AS, and further extending parallel to atrial septum AS. Similarly, second end region 44 includes a plurality of support arms that extend radially outward from the distal end of middle region 46, curving away from middle region 46 into the right atrium, then curving back toward atrial septum AS, and further extending parallel to atrial septum AS. In addition, middle portion 46 of passage device 40 may be expanded to a selected shape and size as described above with reference to passage device 10.

As described above, passage device 40 may be designed to anchor an additional medical device within the heart wall. Specifically, as illustrated in FIG. 4B, passage device 40 may be coupled to left atrial blood pressure sensor 49 for measuring blood pressure within the left atrium. Additionally, sensor 49 may effectively plug passage 48 of passage device 40 such that no blood flow is permitted across passage device 40. As will be understood by a person having ordinary skill in the art, various medical devices may be coupled within the lumen of middle region 46 of passage device 40, e.g., a septal occluder, an open atrial septal shunt, a valved atrial septal shunt, or a blood pump.

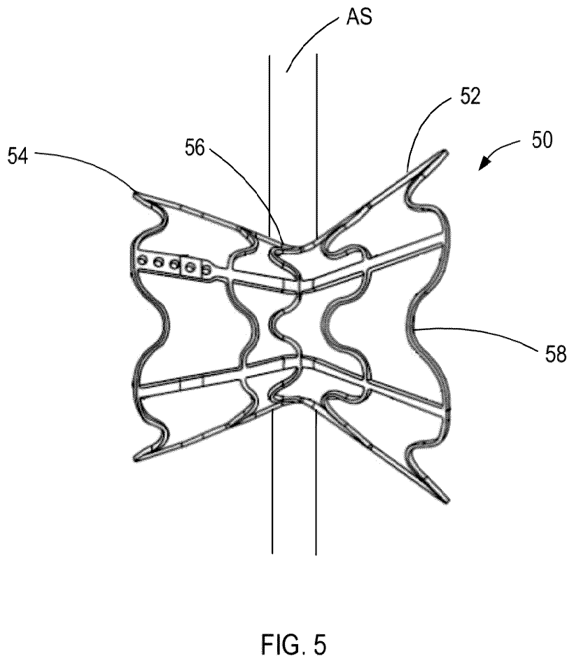

Referring now to FIG. 5, the passage device may be constructed similar to the differential pressure regulating device disclosed in U.S. Pat. No. 10,076,403 to Eigler, assigned to the assignee of the instant application, the entire contents of which is incorporated herein by reference. Specifically, passage device 50 of FIG. 5 has an hourglass shape, and first end region 52 and second end region 54 are integrally formed with middle region 56. For example, first end region 52, second end region 54, and middle region 56 are formed by a plurality of longitudinal struts interconnected by a plurality of circumferential sinusoidal struts. Further, at least one of first end region 52 or second end region 54 may have at least one of a conical or bell shape. Passage device 50 further may include a layer of biocompatible material disposed on at least middle region 56. In addition, middle portion 56 of passage device 50 may be expanded to a selected shape and size as described above with reference to passage device 10. Moreover, first end portion 52 may be expanded such that it extends into the left atrium at any angle between zero and 90 degrees, and second end portion 54 may be expanded such that it extends into the right atrium at any angle between zero and 90 degrees, as described above with reference to passage device 10.

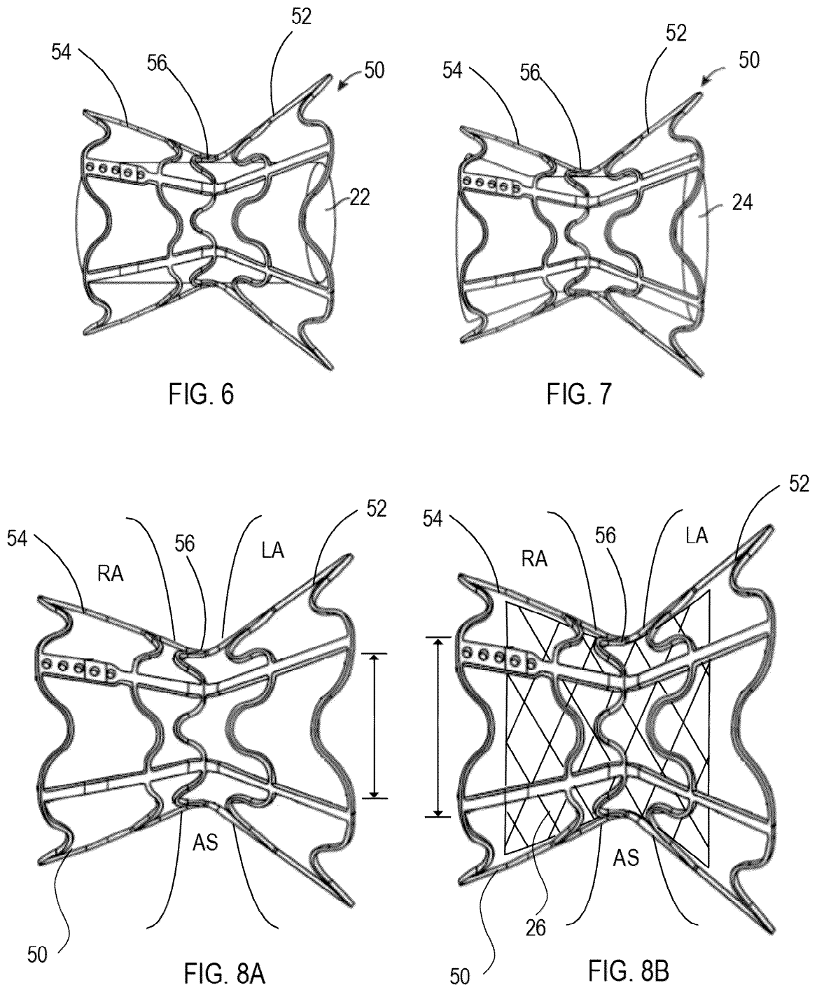

As described above, passage device 50 may be designed to anchor an additional medical device within the heart wall. Specifically, as illustrated in FIG. 6, conduit 22 is registered with respect to the fossa ovalis of the interatrial septum by passage device 50, thereby providing a shunt across the atrial septum. For example, passage device 50 may be an external, unencapsulated bare metal anchor. Conduit 22 may include a separate encapsulated tubular frame or may comprise a tube of solid material, and may include a variety of geometries to achieve specific characteristics as previously described. Passage device 50 and conduit 22 may be physically affixed to each other prior to insertion in the body by mechanical interference, welding, adhesives, or other well-known means, and preferably includes a skirt that prevents bypass flow between passage device 50 and conduit 22. Alternatively, passage device 50 may be delivered across the septum deployed, and then conduit 22 may be inserted through and deployed within passage device 50 and held in place by mechanical interference or expansion with a balloon, or may be self-expanding. The advantages of such a two-part design are two-fold. First, pannus will grow thick only on the outside surface of passage device 50 because the LA and RA ends of conduit 22 are offset from, and thus do not contact, adjacent cardiac structures. Second, the design creates a longest straight channel for high velocity flow, but limits the ability of paradoxical emboli to transit conduit 22 during a transient pressure gradient reversal.

FIG. 7 illustrates another preferred embodiment with benefits similar to that of the shunt of FIG. 6. More specifically, conduit 24 is registered with respect to the fossa ovalis (“FO”) of the interatrial septum by passage device 50, thereby providing a shunt across the atrial septum. Conduit 24 may include flared end regions as described above, e.g., to form an hourglass shape in the deployed state. One of ordinary skill in the art will appreciate that the specific shape of the flared end regions may be conical, parabolic, or horned shaped, and may be present at either or both ends of the shunt device depending on the desired hydraulic properties.

The shunt types depicted in FIG. 6 and FIG. 7, or shunts with similar characteristics that would be apparent to one of ordinary skill in the art, may be particularly applicable to the clinical situation where too large an aperture defect has been created in the FO and where interatrial shunting to treat heart failure is required. Consider the case of a patient with severe mitral regurgitation and poor left ventricular function, where it would be clinically desirable to first perform a repair procedure on the mitral valve, e.g. MitraClip® of mitral annuloplasty by the percutaneous transseptal approach, followed by interatrial shunt placement. These mitral valve procedures currently use a 23Fr I.D. (˜8 mm O.D) guiding catheter to cross the FO. After mitral repair, an anchor with an outer minimal diameter matching the larger aperture defect caused by the prior procedure may be implanted, wherein the conduit as a smaller diameter desirable for shunting (e.g. 5.0 to 6.5 mm). Likewise, such shunts advantageously may be used where, during the transseptal procedure, the FO has been torn, thus creating a larger aperture defect than required for various shunt embodiments. Again, a shunt of the kind described with respect to FIG. 6 or 7 could be used to address such a situation.

Referring now to FIGS. 8A and 8B, additional alternative embodiments are described, where passage device 50 is positioned within the fossa ovalis of the atrial septum as described above, and an expandable metal stent is subsequently placed within the lumen of passage device 50 and expanded to enlarge the cross-sectional area at middle region 56 of passage device 50, for example, after passage device 50 has been chronically deployed. As illustrated in FIG. 8A, passage device 50 is first positioned within a puncture of the atrial septum AS such that first end region 52 extends within left atrium LA, and second end region 54 extends within right atrium RA. Subsequently, as illustrated in FIG. 8B, expandable stent 26 may be deployed within the lumen of passage device 50. Stent 26 may be balloon-expandable or self-expanding. Stent 26 may be an unencapsulated bare metal mesh stent. In accordance other aspects of the present invention, stent 26 may be a drug-eluting mesh stent or an encapsulated mesh stent. In addition, stent 26 may include flared end regions to form an hourglass shape in the deployed state and conform to the shape of passage device 50.

By comparing FIGS. 8A and 8B, introduction of expandable stent 26 within the lumen of passage device 50 causes the diameter at middle region 56 of passage device 50 to increase over time. For example, stent 26 may be self-expanding upon deployment, or an inflatable balloon may be positioned within the lumen of stent 26 and inflated to expand stent 26, and consequently, passage device 50. The balloon is then removed and mechanical interference may physically affix stent 26 to passage device 50 within the atrial septum AS. In addition, stent 26 stent may be coupled to passage device 50 such that it may be periodically removed from passage device 50 while passage device 50 remains anchored within atrial septum AS, if necessary, and replaced with another stent, such as when tissue ingrowth interferes with performance of the stent. Further, stent 26 may be removed any time there is a need to pass a catheter or other medical device between the heart chambers. As will be understood by a person having ordinary skill in the art, various medical devices may be coupled within the lumen of middle region 56 of passage device 50, e.g., a septal occluder, a valved atrial septal shunt, a left atrial blood pressure sensor, or a blood pump.

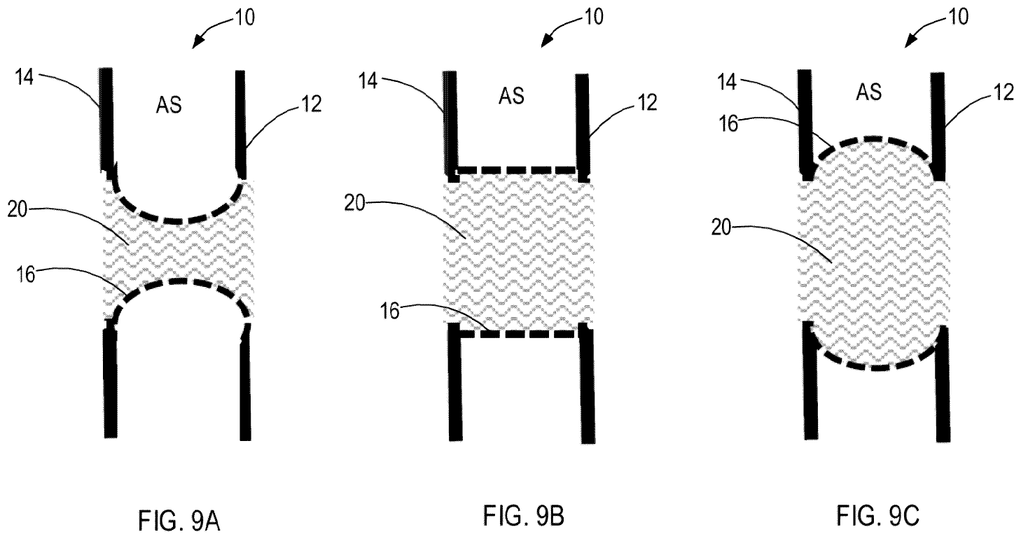

Referring now to FIGS. 9A to 9C, adjustment of the diameter of the passage of a middle region of an exemplary passage device in situ is described. As shown in FIG. 9A, middle region 16 of passage device 10 initially may be concaved inward toward the longitudinal axis of passage device 10, thereby reducing the cross-sectional area of passage 20 of middle region 16. Accordingly, a balloon catheter may be delivered within passage 20 of passage device 10, and inflated to adjust the cross-sectional area across passage 20. For example, as shown in FIG. 9B, the balloon catheter may be inflated until middle region 16 of passage device 10 is linear, parallel to the longitudinal axis of passage device 10. Further, as shown in FIG. 9C, the balloon catheter may be inflated until middle region 16 of passage device 10 is concaved outward away from the longitudinal axis of passage device 10. The size of passage 20 may be selected dependent of the clinical procedure being performed by the surgeon. As will be understood by a person having ordinary skill in the art, other expanding devices may be used to apply force against middle region 16 to thereby adjust the size of passage 20 of passage device 10.

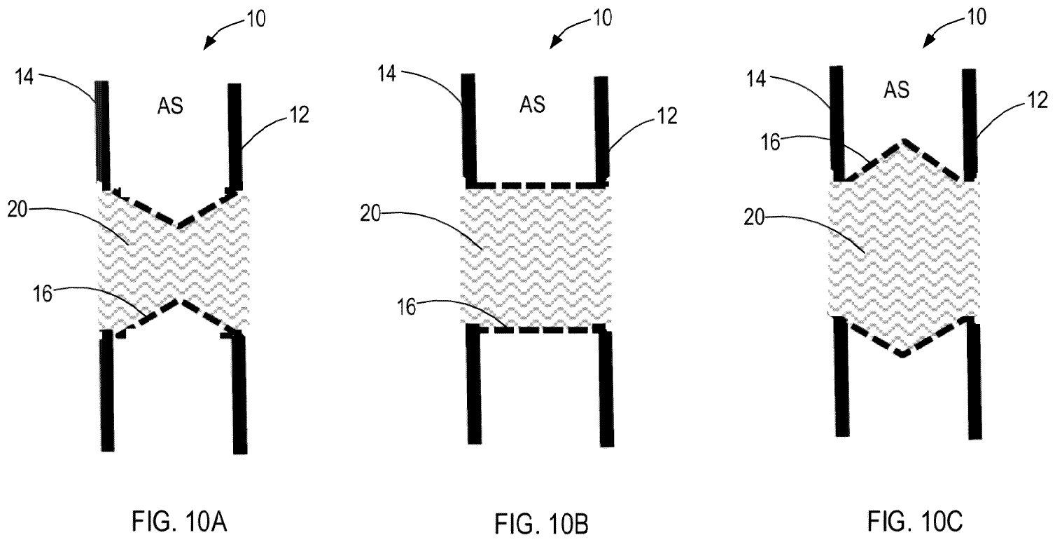

Referring now to FIGS. 10A to 10C, adjustment of the diameter of the passage of a middle region of an alternative exemplary passage device in situ is described. As shown in FIG. 10A, middle region 16 of passage device 10 initially may be pointed inward toward the longitudinal axis of passage device 10, e.g., in a triangular manner, thereby reducing the cross-sectional area of passage 20 of middle region 16. Accordingly, a balloon catheter may be delivered within passage 20 of passage device 10, and inflated to adjust the cross-sectional area across passage 20. For example, as shown in FIG. 10B, the balloon catheter may be inflated until middle region 16 of passage device 10 is linear, parallel to the longitudinal axis of passage device 10. Further, as shown in FIG. 10C, the balloon catheter may be inflated until middle region 16 of passage device 10 is pointed outward away from the longitudinal axis of passage device 10, e.g., in a triangular manner. The size of passage 20 may be selected dependent of the clinical procedure being performed by the surgeon. As will be understood by a person having ordinary skill in the art, other expanding devices may be used to apply force against middle region 16 to thereby adjust the size of passage 20 of passage device 10.

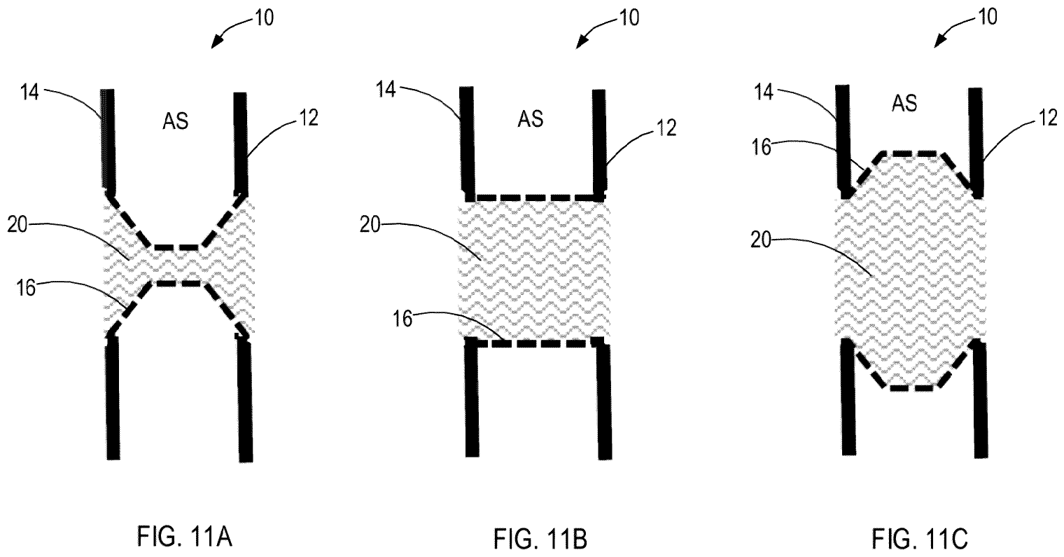

Referring now to FIGS. 11A to 11C, adjustment of the diameter of the passage of a middle region of another alternative exemplary passage device in situ is described. As shown in FIG. 11A, middle region 16 of passage device 10 initially may be trapezoidally pointed inward toward the longitudinal axis of passage device 10, thereby reducing the cross-sectional area of passage 20 of middle region 16. Accordingly, a balloon catheter may be delivered within passage 20 of passage device 10, and inflated to adjust the cross-sectional area across passage 20. For example, as shown in FIG. 11B, the balloon catheter may be inflated until middle region 16 of passage device 10 is linear, parallel to the longitudinal axis of passage device 10. Further, as shown in FIG. 11C, the balloon catheter may be inflated until middle region 16 of passage device 10 is trapezoidally pointed outward away from the longitudinal axis of passage device 10. The size of passage 20 may be selected dependent of the clinical procedure being performed by the surgeon. As will be understood by a person having ordinary skill in the art, other expanding devices may be used to apply force against middle region 16 to thereby adjust the size of passage 20 of passage device 10. In addition, passage device 10 may include one or more sensors for measuring blood flow through passage 20 between the left atrium and the right atrium, such that middle region 16 may be adjusted from responsive to the measured blood flow. For example, the one or more sensors may be at least one of a pressure sensor, ultrasound probe, blood flow sensor, temperature sensor or oxygen saturation sensor.

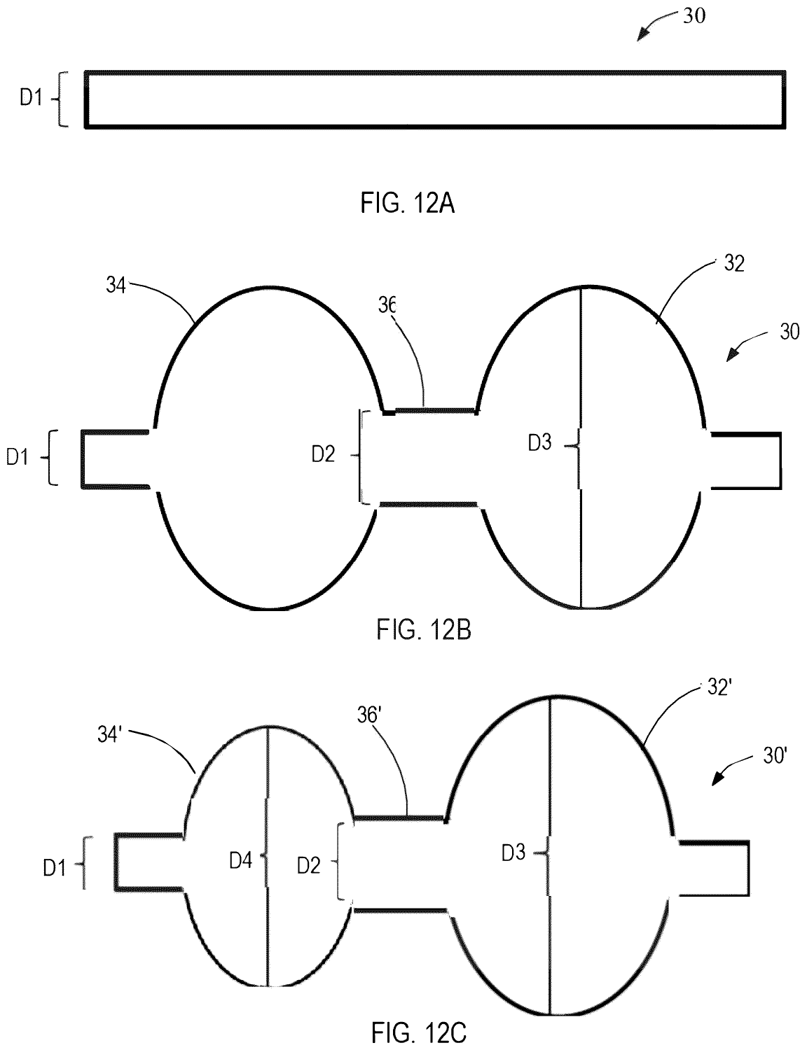

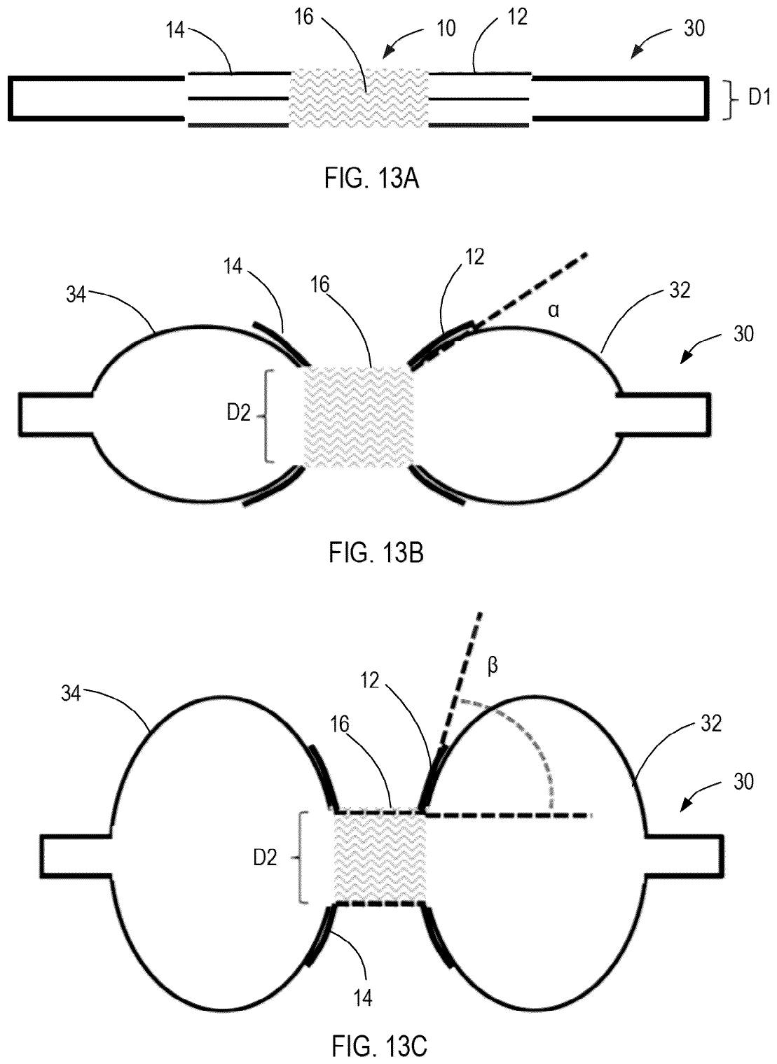

Referring now to FIGS. 12A and 12B, an exemplary dog bone shaped balloon catheter is described. As shown in FIG. 12A, balloon catheter 30 may have an initial diameter D1. For example, D1 may be any size between, e.g., 0.5 mm to 0.5 cm, 0.5 mm to 1 cm, or 0.1 mm to 2 cm. As shown in FIG. 12B, balloon catheter 30 may be inflated such that proximal portion 32 and distal portion 34 expand to have diameter D3, to thereby adjust the angle and cross-sectional area of the first and second end regions of the passage device as described in further detail below with reference to FIGS. 13A to 13C. For example, D3 may be any size between, e.g., 1 mm to 2 cm, 1 mm to 5 cm, or 0.5 mm to 10 cm. In addition, middle portion 36 may be expanded to have diameter D2. For example, D2 may be any size between, e.g., 1 mm to 1 cm, 1 mm to 2 cm, or 0.5 mm to 5 cm. In accordance with another aspect of the present invention, balloon catheter 30 may be formed such that upon inflation, the proximal portion and the distal portion expand to have different diameters from each other. For example, as shown in FIG. 12C, balloon catheter 30′ may be inflated such that proximal portion 32′ expands to have diameter D3, whereas distal portion 34′ expands to have diameter D4. For example, D4 may be any size between, e.g., 1 mm to 2 cm, 1 mm to 5 cm, or 0.5 mm to 10 cm.

Referring now to FIGS. 13A to 13C, balloon catheter 30 may be used to adjust the angle and cross-sectional area of first end region 12, second end region 14, and middle region 16 of passage device 10. As shown in FIG. 13A, balloon catheter 30 is first introduced within passage 20 of passage device 10 in a deflated state after passage device 10 is positioned within the heart wall. As shown in FIG. 13B, balloon catheter may be inflated such that middle region 16 expands to have diameter D2. Accordingly, as first portion 32 and second portion 34 expand upon inflation of balloon catheter 30, first portion 32 and second portion 34 will apply a force against first end region 12 and second end region 14, respectively, thereby causing first end region 12 and second end region 14 to expand and extend into their respective atria at angle α relative to the longitudinal axis of passage device 10. For example, α may be between zero and 45 degrees, preferably 30 to 45 degrees. In addition, as shown in FIG. 13C, balloon catheter 30 may be further inflated such that first portion 32 and second portion 34 expand and apply an additional force against first end region 12 and second end region 14, respectively, thereby causing first end region 12 and second end region 14 to expand and extend into their respective atria at angle β relative to the longitudinal axis of passage device 10. For example, β may be between 45 and 90 degrees.

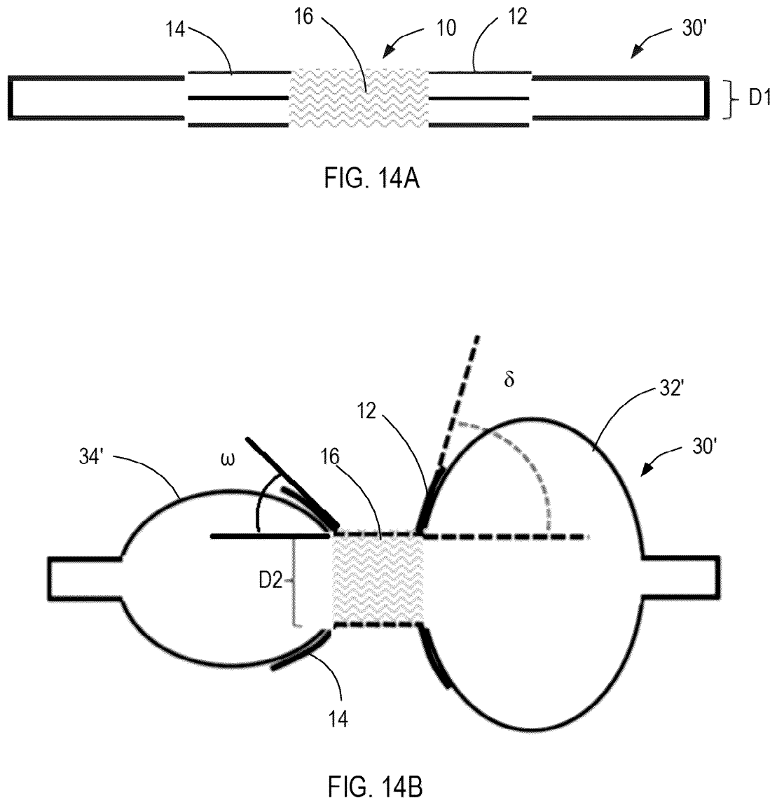

Referring now to FIGS. 14A and 14B, balloon catheter 30′ may be used to asymmetrically adjust the angle and cross-sectional area of first end region 12, second end region 14, and middle region 16 of passage device 10. As shown in FIG. 14A, balloon catheter 30′ is first introduced within passage 20 of passage device 10 in a deflated state after passage device 10 is positioned within the heart wall. As shown in FIG. 14B, balloon catheter may be inflated such that middle region 16 expands to have diameter D2. Accordingly, as first portion 32′ expands upon inflation of balloon catheter 30′, first portion 32′ will apply a force against first end region 12, thereby causing first end region 12 to expand and extend into the left atrium at angle δ relative to the longitudinal axis of passage device 10. For example, δ may be between 45 and 90 degrees. In addition, as second portion 34′ expands upon inflation of balloon catheter 30′, second portion 34′ will apply a force against second end region 14, thereby causing second end region 14 to expand and extend into the right atrium at angle ω relative to the longitudinal axis of passage device 10. For example, ω may be between zero and 45 degrees, preferably 30 to 45 degrees.

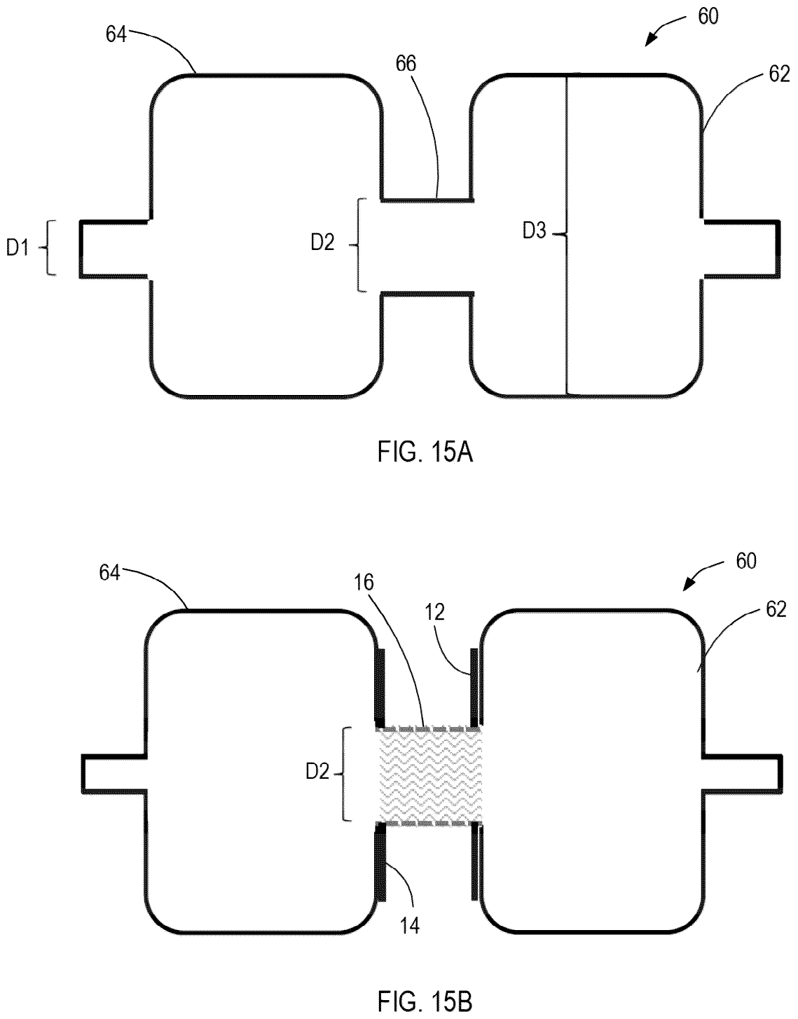

Referring now to FIGS. 15A and 15B, an exemplary quadrilateral dog bone shaped balloon catheter is described. Balloon catheter 60 may be constructed similar to balloon catheter 30, except that first portion 62 and second portion 64 have a quadrilateral shape upon inflation of balloon catheter 60. Accordingly, as shown in FIG. 15A, balloon catheter 60 may be inflated such that proximal portion 62 and distal portion 64 expand to have diameter D3, and middle portion 66 expands to have diameter D2. As shown in FIG. 15B, upon inflation of balloon catheter 60 within passage 20 of passage device 10, first portion 62 and second portion 64 apply a force against first end region 12 and second end region 14, respectively, thereby causing first end region 12 and second end region 14 to expand and extend into their respective atria at an angle of 90 degrees relative to the longitudinal axis of passage device 10. As will be understood by a person having ordinary skill in the art, first and second portions of the balloon catheter may be pre-formed to have various shapes and sizes upon inflation of the balloon catheter to achieve the desired adjustment of the first end region, second end region, and middle region of the passage device.

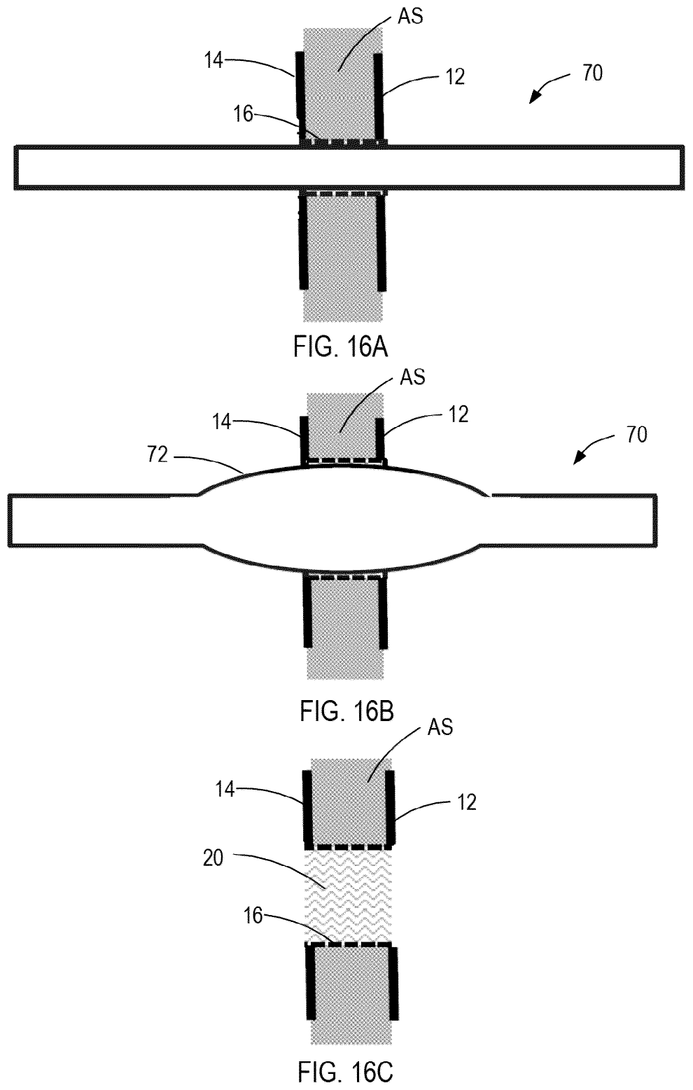

Referring now to FIGS. 16A to 16C, an exemplary balloon catheter for adjusting the size of the passage of the middle region of the passage device in situ is provided. As shown in FIG. 16A, balloon catheter 70 is first introduced through passage 20 of passage device 10 in a deflated state after passage device 10 is deployed within atrial septum AS. Then, as shown in FIG. 16B, balloon catheter 70 is inflated such that expandable portion 72 of balloon catheter 70 expands to a desired size. As expandable portion 72 expands, expandable portion 72 applies a force against middle region 16, thereby causing the diameter of passage 20 of passage device 10 to increase. Balloon catheter 70 may be inflated until passage 20 reaches a desired size dependent on the clinical procedure being performed by the surgeon. Balloon catheter 70 is then removed from within passage 20 of passage device 10, leaving passage 20 at the desired size due to the deformable plastic properties of middle region 16 as shown in FIG. 16C.

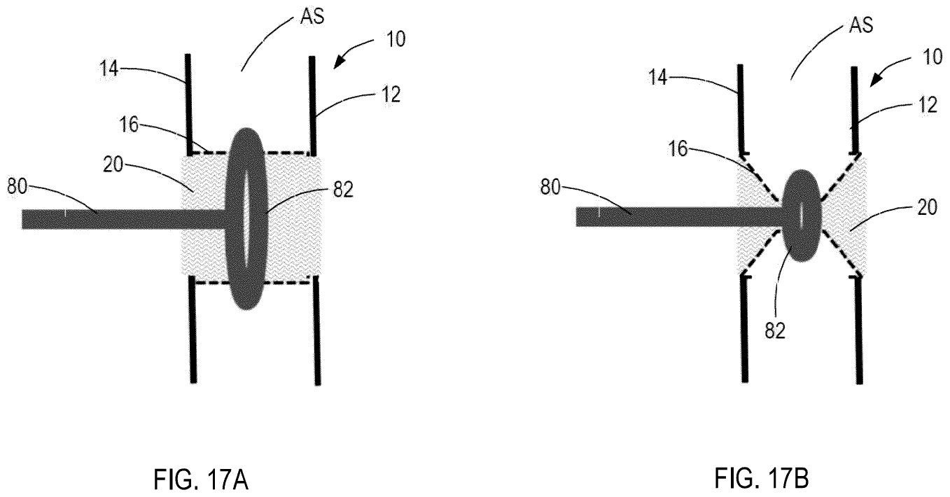

Referring now to FIGS. 17A and 17B, the steps of using a snare to adjust the size of the passage of a passage device in accordance with the principles of the present invention is provided. Snare 80 may be advanced over passage device 10 when passage device 10 is deployed within atrial septum AS such that middle region 16 is positioned within snare hoop 82 of snare 80. As shown in FIG. 17A, snare hoop 82 has an initial diameter such that middle region 16 fits within snare hoop 82 in an uncompressed state. As shown in FIG. 17B, snare 80 may be actuated such that the diameter of snare hoop 82 decreased from the initial diameter to a smaller diameter, thereby causing middle region 16 to compress, and accordingly, passage 20 to decrease in size. For example, snare hoop 82 may be formed of a wire that is exposed through a lumen of a tubular catheter of snare 80, wherein the size of snare hoop 82 may be adjusted by retracting the wire through the lumen of the tubular catheter. As will be understood by a person having ordinary skill in the art, snare 80 may be actuated to selectively adjust the size of snare hoop 82, to thereby control the size of passage 20 of passage device 10, and accordingly, the flowrate across passage device 10. The size of passage 20 also may be adjusted to accommodate clinical procedure tools required by the surgeon. In addition, a balloon catheter may be used in conjunction with snare 80 to ensure that middle region 16 is compressed to the desired passage size. For example, the balloon catheter may be delivered within passage 20 of passage device 10 when middle region 16 is within snare hoop 82, and inflated to the desired passage size. Accordingly, snare 80 may be actuated such that the opening of snare hoop 82 is reduced to conform with the inflated balloon catheter, thereby achieving the desired passage size of middle region 16.

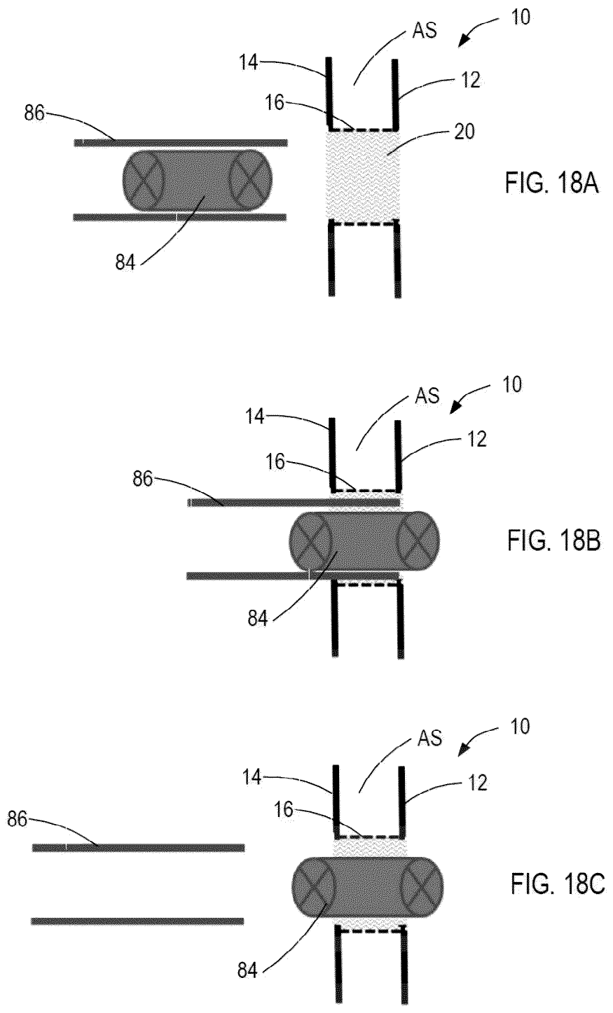

Referring now to FIGS. 18A to 18C, the steps of subsequently placing a medical device within the device of FIG. 1A in accordance with the principles of the present invention is provided. As described above, passage device 10 may be designed to anchor an additional medical device within the heart wall. As shown in FIG. 18A, medical device 84 may be disposed within sheath 86 for delivery to passage device 10. As illustrated in FIG. 18B, sheath 86 is delivered through passage 20 of passage device 10 such that medical device 84 is aligned with middle region 16 of passage device 10. Sheath 86 may then be retracted, leaving medical device 84 positioned within passage 20, as shown in FIG. 18C. Medical device 84 may be coupled to passage device 10 via techniques readily known in the art. Medical device 84 may be, e.g., a septal occluder, an open atrial septal shunt, a valved atrial septal shunt, a left atrial blood pressure sensor, or a blood pump. In addition, medical device 84 may be removed and replaced according to the needs of the patient and the clinical procedure.

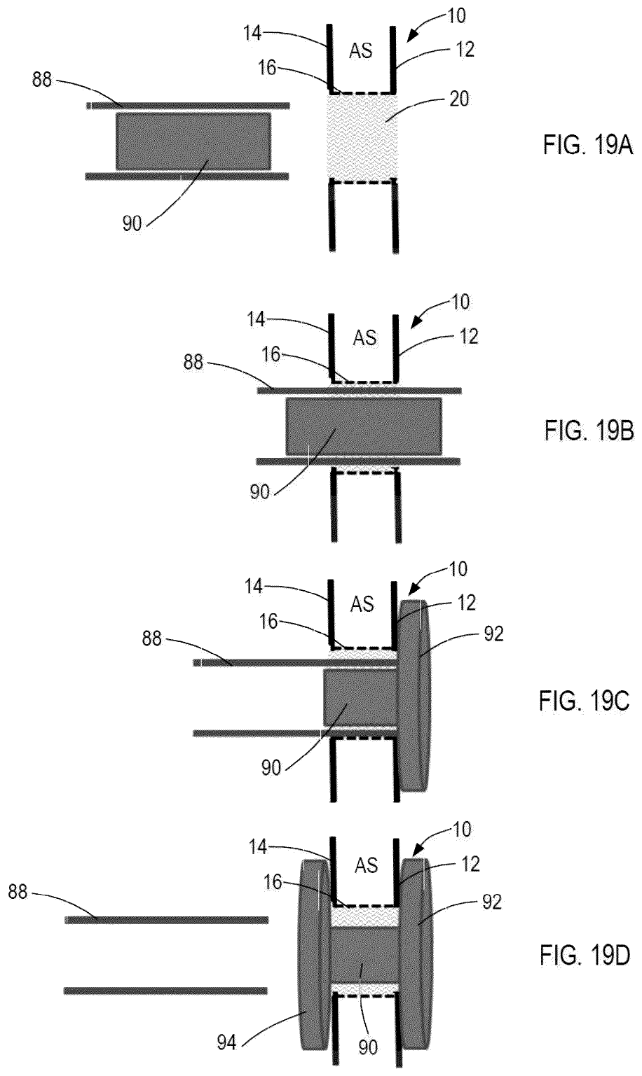

Referring now to FIGS. 19A to 19D, the steps of subsequently placing a medical device having expandable ends within the device of FIG. 1A in accordance with the principles of the present invention is provided. As shown in FIG. 19A, medical device 90 may be disposed within sheath 88 for delivery to passage device 10. Medical device 90 includes first expandable portion 92 sized and shaped to be disposed within the left atrium, and second expandable portion 94 sized and shaped to be disposed within the right atrium upon deployment of medical device 90. As shown in FIG. 19A, first and second expandable portions 92, 94 may be disposed within sheath 88 in a compressed delivery state. As illustrated in FIG. 19B, sheath 88 is delivered through passage 20 of passage device 10 such that medical device 90 is aligned with middle region 16 of passage device 10.

Sheath 88 may then be partially retracted, exposing first expandable portion 92 beyond the opening of sheath 88 such that first expandable portion 92 transitions from the compressed delivery stated to an expanded deployed state within the left atrium, as shown in FIG. 19C. For example, first expandable portion 92 may be self-expanding. Sheath 88 and medical device 90 are positioned within passage 20 of passage device 10 such that first expandable portion 92 of medical device 90 is positioned adjacent first end region 12 of passage device 10 within the left atrium.

As shown in FIG. 19D, sheath 88 may be further retracted, exposing second expandable portion 94 beyond the opening of sheath 88 such that second expandable portion 94 transitions from the compressed delivery stated to an expanded deployed state within the right atrium. For example, second expandable portion 94 may be self-expanding. In the expanded state, first and second expandable portions 92, 94 of medical device 90 sandwich passage device 10 within atrial septum AS, thereby assisting anchoring of medical device 90 within passage device 10 at atrial septum AS.

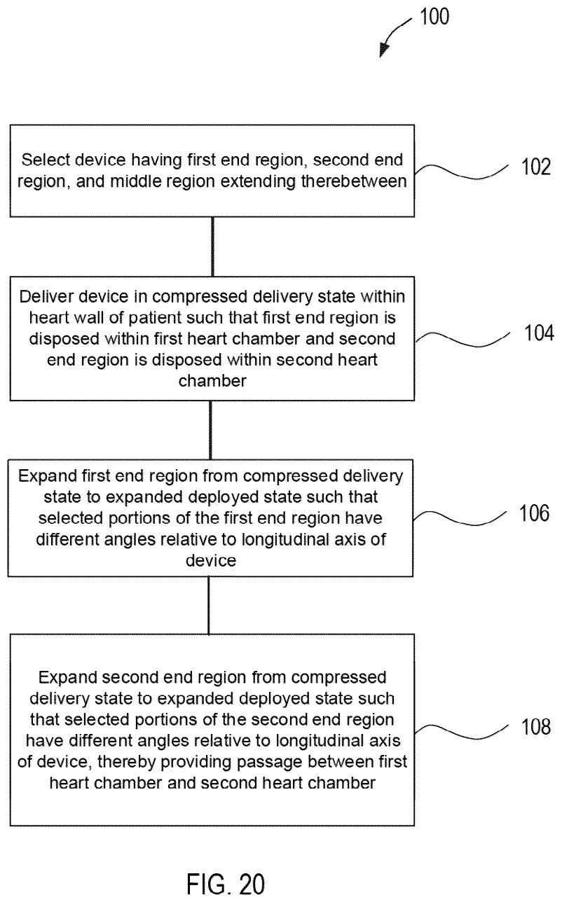

In accordance with another aspect of the present invention, exemplary method 100 for transitioning the end regions of a passage device from a compressed delivery state to an expanded deployed state in accordance with the principles of the present invention is provided. As shown in FIG. 20, at step 102, a passage device is selected, e.g., any of the passage devices described above. The passage device includes a first end region, a second end region, and a middle region extending between the first and second end regions. The passage device may be transitioned to a compressed delivery state and disposed within a delivery sheath for percutaneous delivery. At step 104, the passage device is delivered to the target site, e.g., an atrial septum between left and right atria, in the compressed delivery state. The sheath may be retracted to deploy the passage device at the atrial septum such that the first end region is disposed within the left atrium, the second end region is disposed within the right atrium, and the middle region of the passage device is disposed within an opening in the atrial septum.

At step 106, a balloon catheter, e.g., any of the balloon catheters described above, or any other suitable expanding tool, is introduced through the lumen of the passage device. The balloon catheter is then inflated to expand the first end region from the compressed delivery state to an expanded deployed state in the left atrium such that selected portions of the first end region have different angles relative to the longitudinal axis of the passage device. For example, the balloon catheter may have multiple inflatable portions, each in fluid communication with its own fluid lumen of the balloon catheter, so that the selected portions of the first and second end regions may be expanded to the desired angle for that selected portion.

At step 108, inflation of the balloon catheter causes the second end region to expand from the compressed delivery state to an expanded deployed state in the right atrium such that selected portions of the second end region have different angles relative to the longitudinal axis of the passage device. As will be understood by a person having ordinary skill in the art, step 106 and step 108 may occur simultaneously upon inflation of the balloon catheter. In addition, the first and second end regions may be expanded to extend within their respective atria at the same angle relative to the longitudinal axis of the passage device. The balloon catheter may then be removed such that the middle region of the passage device maintains its adjusted size.

While various illustrative embodiments of the invention are described above, it will be apparent to one skilled in the art that various changes and modifications may be made herein without departing from the invention. For example, the inventive devices and methods may be used following, e.g., a mitral valve (“MV”) repair procedure, where the passage device is placed within the mitral valve having a desired passage size. MV repair may result in a larger than optimal orifice, and thus the adjustable passage device may be used to optimize flow across the MV, which may be readjusted later as described above in accordance with the principles of the present invention.

What is claimed:

1. A device for modifying blood flow within a patient, the device comprising:
a shunt configured to transition between a compressed delivery state and an expanded deployed state, the shunt comprising a middle region, a first end region extending from a first end of the middle region and configured to be deployed in a first body cavity of the patient, a second end region extending from a second end of the middle region and configured to be deployed in a second body cavity of the patient, and a lumen extending therethrough and defining the passage between the first and second body cavities,
wherein select portions of the first and second end regions are configured to be independently, selectively deformable to different angles relative to a longitudinal axis of the shunt upon application of one or more forces thereto, the different angles of the select portions of the first and/or second end regions configured to be maintained in the expanded deployed state upon release of the one or more forces to thereby change a flow rate of blood through the passage defined by the lumen between the first and second body cavities, thereby changing a coefficient of discharge of the shunt.

2. The device of claim 1, wherein the first body cavity is defined by a first heart chamber and the second body cavity is defined by a second heart chamber, and
wherein, in the expanded deployed state, the middle region is configured to be disposed within a heart wall between the first and second heart chambers.

3. The device of claim 2, wherein the first heart chamber is a left atrium and the second heart chamber is a right atrium, and wherein the shunt is configured to permit blood flow through the passage between the left atrium and the right atrium.

4. The device of claim 1, wherein the first body cavity and the second body cavity are defined by one or more blood vessels, and wherein the shunt is configured to permit blood flow through the passage.

5. The device of claim 1, wherein the first body cavity is defined by a blood vessel and the second body cavity is defined by a heart chamber, and wherein the shunt is configured to permit blood flow through the passage between the blood vessel and the heart chamber.

6. The device of claim 1, wherein the first and second end regions comprise a plastically deformable material.

7. The device of claim 1, wherein the first and second end regions are configured to be independently, selectively deformable to different angles between zero and 90 degrees relative to the longitudinal axis of the shunt.

8. The device of claim 1, wherein the middle region is configured to be adjusted from a first state having a first diameter to a second state having a second diameter different from the first diameter.

9. The device of claim 8, wherein the middle region comprises a plastically deformable material.

10. The device of claim 8, further comprising:
one or more sensors configured to measure blood flow through the lumen of the shunt between the first and second body cavities,
wherein the middle region is configured to be adjusted from the first state to the second state responsive to the measured blood flow.

11. The device of claim 1, wherein the shunt comprises an hourglass shape in the expanded deployed state.

12. The device of claim 1, wherein at least one of the first or second end regions comprises at least one of a conical or bell shape.

13. The device of claim 1, wherein the first and second end regions are integrally formed with the middle region.

14. The device of claim 1, wherein the first and second end regions and the middle region comprise a plurality of longitudinal struts interconnected by a plurality of circumferential sinusoidal struts.

15. A system for providing a passage between adjacent body cavities within a patient, the system comprising:
the device of claim 1; and
one or more expansion devices configured to be actuated to apply the one or more forces to the first and/or second end regions to independently, selectively deform select portions of the first and/or second end regions to different angles relative to the longitudinal axis of the shunt.

16. The system of claim 15, wherein the one or more expansion devices comprise one or more different sized balloon catheters configured to be inflated within the lumen of the shunt to thereby apply the one or more forces to the first and/or second end regions.

17. A method for modifying blood flow within a patient, the method comprising:
selecting a shunt comprising a middle region, a first end region extending from a first end of the middle region, a second end region extending from a second end of the middle region, and a lumen extending therethrough;
delivering the shunt in a compressed delivery state through the patient's vasculature such that the first end region is disposed within a first body cavity, the second end region is disposed within a second body cavity, the middle region is positioned within a tissue between the first and second body cavities, and the lumen defines the passage between the first and second body cavities; and
applying one or more forces to the first and/or second end regions to independently, selectively deform select portions of the first and/or second end regions to different angles relative to a longitudinal axis of the shunt,
wherein, upon release of the one or more forces, the different angles of the select portions of the first and/or second end regions are maintained in the expanded deployed state to thereby change a flow rate of blood through the passage defined by the lumen between the first and second body cavities, thereby changing a coefficient of discharge of the shunt.

18. The method of claim 17, wherein delivering the shunt in the compressed delivery state through the patient's vasculature comprises delivering the shunt in the compressed delivery state through the patient's vasculature such that the first end region is disposed within a first heart chamber, the second end region is disposed within a second heart chamber, the middle region is positioned within a heart wall between the first and second heart chambers, and the lumen defines the passage between the first and second heart chambers.

19. The method of claim 17, wherein delivering the shunt in the compressed delivery state through the patient's vasculature comprises delivering the shunt in the compressed delivery state through the patient's vasculature such that the first end region is disposed within a blood vessel, the second end region is disposed within a heart chamber, the middle region is positioned within a tissue between the blood vessel and the heart chamber, and the lumen defines the passage between the blood vessel and the heart chamber.

20. The method of claim 17, wherein applying one or more forces to the first and/or second end regions comprises actuating one or more expansion devices to apply the one or more forces to the first and/or second end regions to independently, selectively deform select portions of the first and/or second end regions to different angles relative to the longitudinal axis of the shunt.
